(19) United States
(12) Patent Application Publication
(10) Pub. No.: US 2015/0125533 A1
(43) Pub. Date: May 7, 2015

(54) SINGLE-DOMAIN ANTIBODIES AND GRAPHENE COATED MAGNETIC METAL NANOPARTICLES CONJUGATE AND METHODS FOR USING THE SAME

(71) Applicants: Mohamed Sallam, New Cairo (EG); Suher Zada, New Cairo (EG); Ibrahim Rabie, Cairo (EG); Adham Ramadan, New Cairo (EG)

(72) Inventors: Mohamed Sallam, New Cairo (EG); Suher Zada, New Cairo (EG); Ibrahim Rabie, Cairo (EG); Adham Ramadan, New Cairo (EG)

(73) Assignee: AMERICAN UNIVERSITY IN CAIRO, New Cairo (EG)

(21) Appl. No.: 14/235,415

(22) PCT Filed: Jul. 25, 2012
(86) PCT No.: PCT/IB2012/001846

§ 371 Date: Apr. 28, 2014

Related U.S. Application Data
(60) Provisional application No. 61/511,451, filed on Jul. 25, 2011.

(51) Int. Cl. A61K 47/48; A61K 33/26; A61K 45/06; A61K 47/02

(52) U.S. Cl. CPC A61K 47/48384; A61K 47/02; A61K 33/26; A61K 45/06; A61K 47/48861
USPC 424/490; 424/178.1; 427/2.11

(57) ABSTRACT

Single-domain antibodies and graphene coated magnetic metal nanoparticles conjugate and methods for using the same. In certain aspects, graphene coated nanoparticles comprise a targeting moiety, such as a nanobody, and may be used for various targeted therapies (e.g., diseased tissues and cancer). Methods for using magnetic nanoparticles for treatment of parasitic infections are also provided.

This application claims the benefit of U.S. Provisional Patent Application No. 61/511,451, filed Jul. 25, 2011, which is incorporated herein by reference in its entirety.

### BACKGROUND OF THE INVENTION

1. Field of the Invention

The present invention relates generally to the fields of single domain nanobodies, nanotechnology, medicine and nanoparticle diagnosis and therapy.

2. Description of Related Art

Magnetic nanoparticles are employed in numerous areas of medical studies, for instance contrast agents for magnetic resonance imaging of biological tissues and processes and colloidal mediators for magnetic hyperthermia of diseased tissues, parasitic infections, and cancer. Their chemical and physical stability, biocompatibility and superior targeting specificity are the most crucial factors for their use for various in vivo applications.

### SUMMARY OF THE INVENTION

In a first embodiment the invention provides a nanoparticle or a population of nanoparticles comprising (a) a core comprising a magnetic pure and/or hybrid metal(s) and (b) a graphene coating surrounding the core. For example, a particle may comprise a core comprising iron, iron-platinum, cobalt, nickel or an oxide of any of the foregoing. In certain aspects, the core is 60%, 70%, 80%, 90% or 95% of the metal (e.g., iron) by weight. In further aspects, the core is greater than 60%, 70%, 80%, 90% or 95% by weight non-oxidized metal or is substantially free of oxidized metal. For example, the core may comprise less than about 20%, 10%, 5%, 3% or 1% by weight metal oxide, such as iron oxide. In further aspects the nanoparticle comprises a targeting moiety conjugated directly or indirectly (e.g., attached via an intermediate polymer) to the graphene coating. As used herein “conjugated” refers to an association between two elements (such as a graphene layer and a targeting moiety) which may be covalent or noncovalent.

Nanoparticles in according with the embodiments can be produced in a wide range of sizes, such a population having an average diameter from about 10 nm to about 500 nm, about 10 nm to about 300 nm, 10 to about 150 nm, about 20 to about 40 nm or about 30 nm. In certain aspects, populations of nanoparticles are substantially mono-disperse and have an average diameter of about 25 nm to about 35 nm.

Nanoparticles of the embodiments comprise, in certain aspects, a single graphene or multilayered graphitic carbon coating. For example, the graphene coating can form a fullerene structure around the core of particles (i.e., a fullerene lattice encapsulating a magnetic metal core). In certain aspects, a graphene coating is deposited on the particle(s) by microwave arc discharge, and radio frequency-catalytic chemical vapor deposition (RF-cCVD) see, e.g., Liang et al. 2008, and Biris et al. 2010, incorporated herein by reference. Thus, a graphene coating may comprise 1, 2, 3, or more individual layers of graphene.

In a second embodiment there is provided pharmaceutical composition comprising a plurality of nanoparticles according to the embodiments and pharmaceutically acceptable carrier.

In certain aspects, nanoparticles according to the embodiments comprise a further coating (e.g., covalently or non-covalently attached to the graphene coating) such as a polymer coating. In some aspects, the polymer coating may be used to attach a functional element to particles, such as a targeting moiety or therapeutic agent. Examples of such coatings include, but are not limited to, polyglutamic acid, polyacrylic acid, polypropylene glycol, copolymers of linear and branched polyethylene glycol and polypropylene glycol, polylysine, polyvinyl alcohol, human serum albumin, bovine serum albumin, hyaluranic acid, polyethyleimine (PEI), polyvinylprrolidone (PVP) or polyethylene glycol (PEG).

In a further embodiment a method for making nanoparticles according to the invention is provided comprising (a) reducing a metal salt (e.g., an iron salt) to form a magnetic metal nanoparticle and (b) depositing a graphene coating on the particle by microwave arc discharge. In certain aspects, steps (a) and (b) are performed concomitantly or essentially simultaneously. Likewise, in certain aspects, steps (a) and (b) are performed in the same reaction vessel. In some cases, nanoparticle production is performed in at reduced oxygen concentrations, such as under inert gas protection, to prevent oxidation of the metal core of the particles. In yet further aspects, nanoparticle production methods comprise an additional step of: coating the nanoparticle with a polymer (e.g., a polyglutamic acid); attaching a targeting moiety to the nanoparticle; attaching a targeting moiety and/or therapeutic agent to the nanoparticle; and/or purifying the nanoparticles (e.g., by size exclusion chromatography or by using the magnetic properties of the particles for purification). Thus, in certain embodiments, the invention provides a nanoparticle produced by the foregoing methods.

In still a further embodiment the invention provides a method of treating a subject comprising (a) administering nanoparticles comprising a magnetic metal core; a graphene coating; a targeting moiety and a therapeutic agent to a subject in a amount effective to treat the subject. In yet a further embodiment, a method for treating a subject is provided comprising (a) administering nanoparticles comprising a magnetic metal core; a graphene coating and a targeting moiety to a subject; and (b) applying an alternating current field to the subject, wherein the amount of nanoparticles administered to the subject and the alternating current field applied to the subject are together effective to produce localized hyperthermia in the subject (and affect the therapy). For example, methods according to the embodiments can be used to treat a bacterial infection, a viral infection, a parasite infection, an autoimmune disease or a cell hyperproliferative disease (e.g., cancer).

In still a further embodiment a method of treating a subject is provided comprising: (a) administering nanoparticles comprising a magnetic metal core; a graphene coating and a targeting moiety to a subject; (b) applying a first magnetic field (e.g., a static magnetic field) to the subject, wherein the field applied to the subject is effective to promote accumulation of nanoparticles in a localized region; and (c) applying an alternating current field to the subject, wherein the amount of nanoparticles administered to the subject and the alternating current field applied to the subject are together effective to produce localized hyperthermia in the subject. In some aspects, for example, the first magnetic field strength is about 0.2 to 0.5 T. In further aspects, the alternating current field strength is about 0.5 to 2.0 T and the frequency is about 85 to 110 kHz (e.g., a field strength of about 1.5 T and the frequency is about 85 to 110 kHz).

Thus, in a specific embodiment, a method for treating a parasitic infection (e.g., Schistosomiasis, Fascioliasis, and Filariasis) is provided comprising (a) administering to a subject nanoparticles comprising a magnetic metal core; and a parasite targeting moiety; and (b) applying an alternating current field to the subject, wherein the amount of nanoparticles administered to the subject and the alternating current field applied to the subject are together effective to produce hyperthermia at a site of parasite infection in the subject (e.g., to produce hyperthermia sufficient to damage or kill the parasite). In preferred aspects, nanoparticles for use in such methods comprise a graphene coating as detailed herein. In still further aspects a parasite targeting moiety is a moiety (e.g., and monoclonal antibody or a nanobody) that binds to an antigen in the luminal gut of the parasite. Examples of such gut antigens include, but are not limited to Capthesin B or Capthesin L protein.

In further aspects a nanoparticle of the embodiments comprises one or more additional functional elements attached to, or associated with, its surface. For example, a nanoparticle can comprise a targeting moiety, a targeting ligand, a therapeutic agent, an imaging agent, a peptide, an antibody, a nucleic acid, a small molecule, a polymer or a combination thereof. In certain aspects the functional element (e.g., a targeting moiety) is covalently or non-covalently attached to the nanoparticle. Examples of therapeutic agents for use according to the embodiments include without limitation radiotherapeutic agents, therapeutic hormones, chemotherapeutic agents, toxins (targeted by the nanoparticle), antibiotics, antivirals and antiparasitic medicines and nanobodies. Examples of nucleic acids for conjugation to a nanoparticle include, but are not limited to, an aptamer (e.g., a targeting aptamer), a DNA expression vector, a mRNA, a shRNA, a siRNA, a miRNA or an antisense RNA.

In still further aspects, nanoparticles according to the embodiments comprise a targeting moiety such as an apatmer, ligand, or antibody. As used herein, an “antibody” means an antibody-like molecule (e.g., an anticalin), a Fc portion, a Fab, a Fab2, a ScFv, a single domain antibody or a nanobody. For example, the nanobody can be antigen-specific VHH (e.g., a recombinant VHH) from a camelid IgG2 or IgG3. Methods for producing such antibodies are provided in U.S. Patent Publn. Nos. 20060211088, 20050037421 and 20100021384, each incorporated herein by reference. In certain aspects, a targeting moiety binds to a particular cell of a subject (e.g., an immune cell or a cancer cell). In other aspects the targeting moiety binds an element (e.g., a protein, glycoprotein or lipoprotein molecule) of a foreign organism, such as bacteria, a virus or a parasite. In certain specific aspects the targeting moiety binds to parasite gut antigen such as a Capthesin B or Capthesin L.

In accordance with certain embodiments nanoparticles are used as therapeutics, for example, in administration of hyperthermia therapy. As used herein “hyperthermia” refers to an induced localized heating at an in vivo site. For example, magnetic nanoparticles can be used to mediate hyperthermia by application of an alternating current field. Conventional alternating current field-based devices such as RF heating, inductive heating, microwave-based procedures and ultrasound can be used to induce hyperthermia. For example methods for using low-field MRI for hyperthermia therapy have been described in U.S. Patent No. 20100292564, incorporated herein by reference. In certain aspects, an alternating current of about 50 Hz to about 5 MHz (e.g., 85 kHz to about 110 kHz) is a applied to a magnetizing coil to induce hyperthermia. Likewise, an alternating magnetic field having a strength of about 2 mT to about 80 mT can be employed according to the embodiments. Thus, in certain aspects, a hyperthermia therapy is applied at a proper predetermined frequency to achieve required penetration depth, sufficient predetermined intensity and predetermined exposure time to achieve a local temperature or at least or about 45° C., 50° C., 55° C., 60° C., 65° C. or 70° C.

As detailed above, in certain aspects, the targeting moiety can be defined as a parasite targeting moiety, such as a nanobody that binds to a parasite specific antigen (e.g., a gut antigen in a parasite). Example parasites that can be targeted by such nanoparticles include, but are not limited to, Trematode flukes, such as Fasciolopsis buski, Fasiola hepatica, Fasiola giganta, Opisthorchis sinesis, Paragonimus westermani and Schistosoma species (e.g., Schistosoma mansoni), Cestode worms, such as Taenia species, Diphyllobothrium latum, Echinococcus species or Hymenolepsis species; Nematodes, such as Enterobius vermicularis, Ascaris lumbricoides, Toxocara species, Trichuris trichiura, Ancylostoma duodenale, Necator americanus, Ancylostoma braziliense, Strongyloides stercoralis, Trichinella spiralis, Wuchereria bancrofti, Brugia malayi, Loa loa, Mansonella species, Onchocerca volvulus, Dirofilaria immitis, or Dracunculus medinensis, or Protozoa, such as, Plasmodium species, Babesia species, Trypanosoma species, Leishmania species, Toxoplasma species, Sarcocytis species, Acanthamoeba species, Balamuthia species or Naegleria species.

A nanoparticle or nanoparticle formulation according to the embodiments may be administered, for example, intravenously, intradermally, intraarterially, intraperitoneally, intralesionally, intracranially, intraarticularly, intraprostaticaly, intrapleurally, intratracheally, intravitreally, intravaginally, intrarectally, intratumorally, intramuscularly, intraperitoneally, subcutaneously, subconjunctival, intravesicularlly, mucosally, intrapericardially, intraumbilically, intraocularally, intrathecally, orally, locally, by injection, by infusion, by continuous infusion, by localized perfusion bathing target cells directly, via a catheter, or via a lavage. For example, the composition may be administered by injection or oral administration.

To have a better therapeutic benefit, the nanoparticle or nanoparticle formulation may be administered in combination with at least an additional agent such as a radiotherapeutic agent, a hormonal therapy agent, an immunotherapeutic agent, a chemotherapeutic agent, a cryotherapeutic agent and/or a gene therapy agent.

It is contemplated that any method or composition described herein can be implemented with respect to any other method or composition described herein. Accordingly, aspects of the invention discussed in the context of methods for producing are equally applicable to a method of producing and vise versa.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Diagram depicts an example multilayer graphene coated magnetic nanoparticle according to the embodiments.

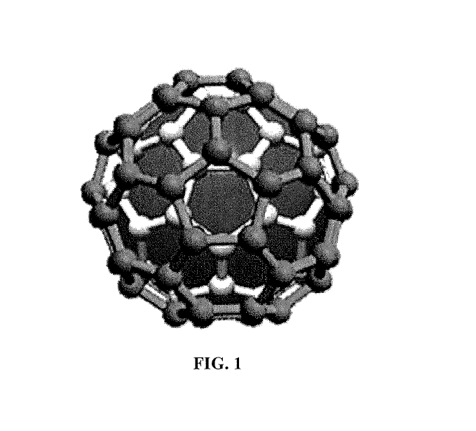

FIG. 2: A schematic diagram which depicts the domain structure of classical antibodies and dromedary IgG 1 (upper panel) versus the heavy chain only antibodies of dromedary IgG2 and IgG3.

FIG. 3: A schematic diagram which depicts an example protocol for producing antigen-specific recombinant VHH.

FIG. 4: ^{99m}Tc-labeled nanobody coated particles in solution in a petri-dish. Dynamic gamma camera imaging was achieved at 1 s per frame in a 5 min acquisition.

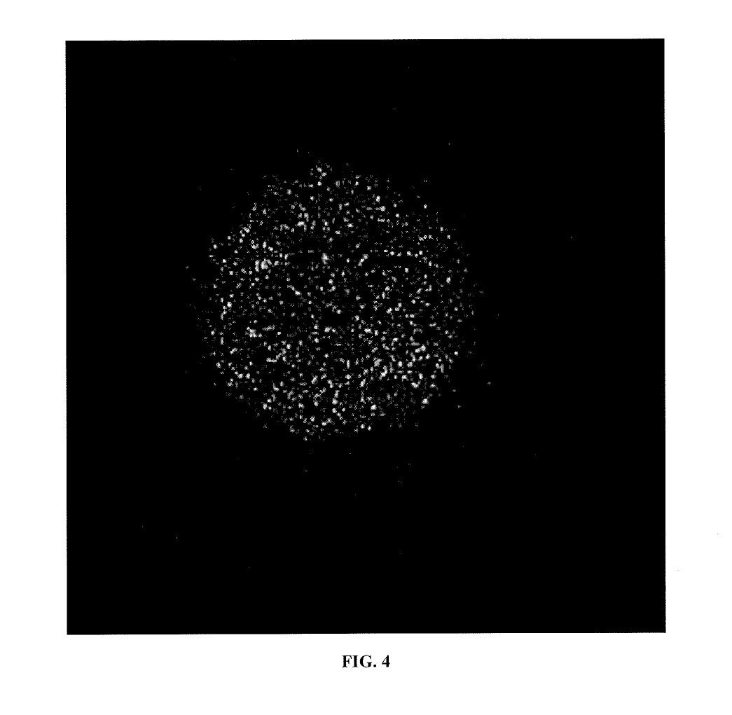

FIG. 5: ^{99m}Tc-labeled nanobody coated particles of FIG. 4 imagined as a described after a magnet was applied at 1 min. Results of the study showed strong focalization of the radiolabeled nanobody coated particles.

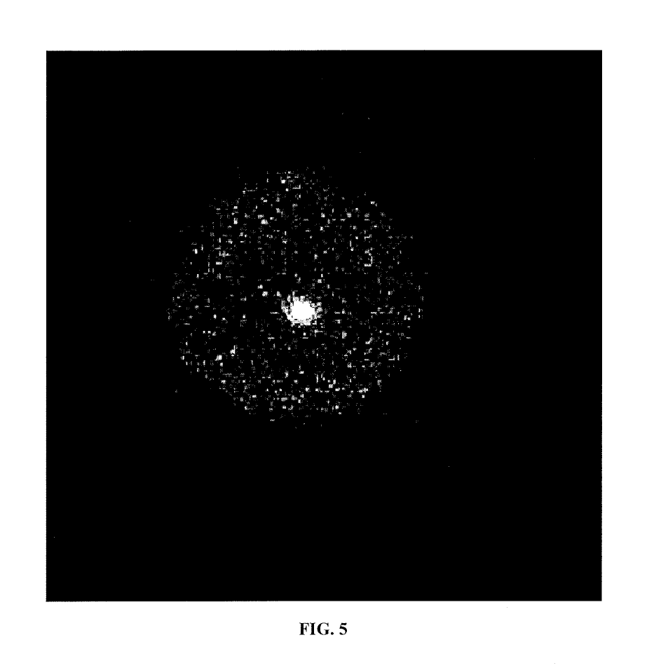

FIG. 6: Graph shows a quantitative analysis of the magnetic focalization studies depicted in FIGS. 4 and 5.

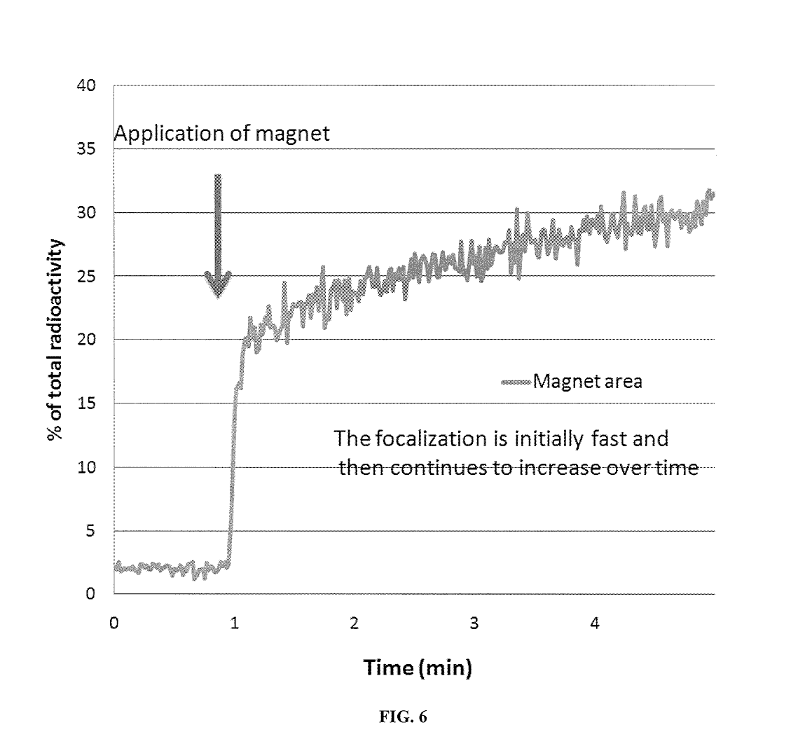

FIG. 7: ^{99m}Tc-labeled nanobody coated particles tube experiment. ^{99m}Tc-labeled nanobody coated particles in solution in a falcon tube. Dynamic gamma camera imaging was achieved with 1 s per frame in a 5 min acquisition.

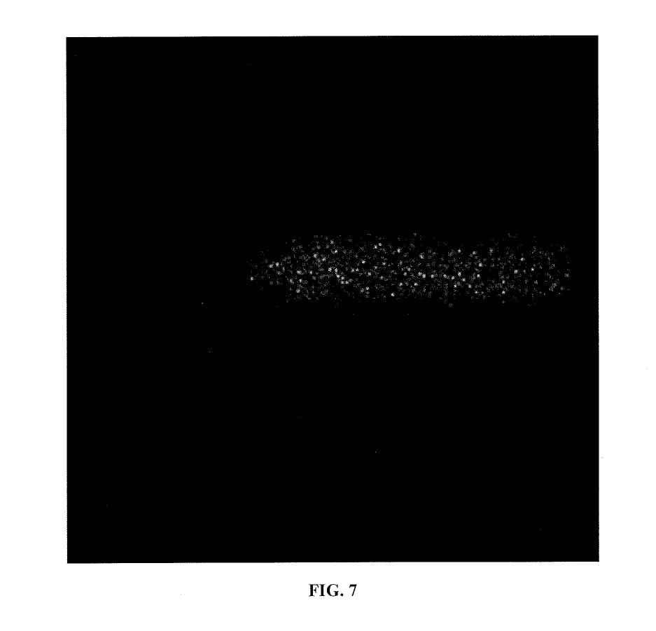

FIG. 8: ^{99m}Tc-labeled nanobody coated particles of FIG. 7 imaged as described after a magnet was applied for 1.5 min. Results show a hypointense area in the region around the focal point (region of the strongest magnetic field).

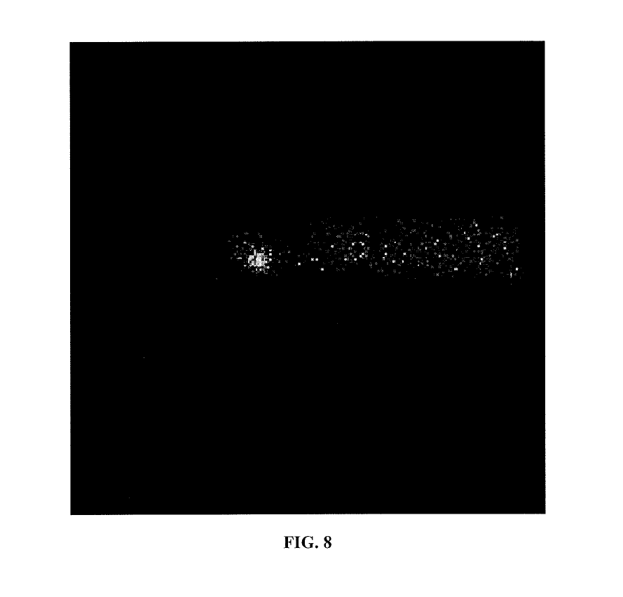

FIG. 9: Graph shows a quantitative analysis of the magnetic focalization studies depicted in FIG. 6.

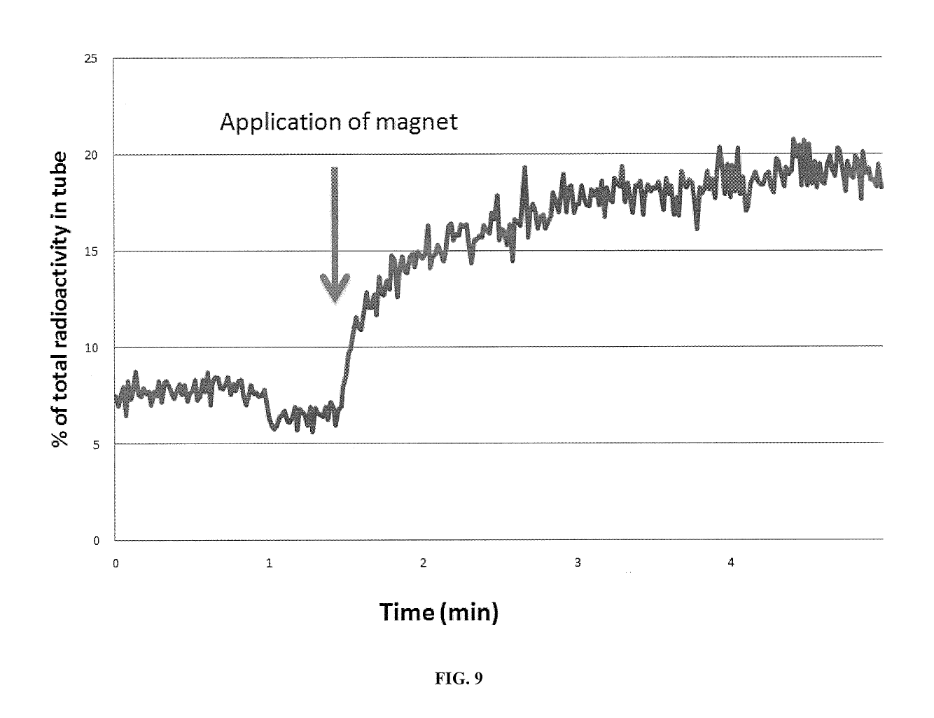

### DESCRIPTION OF ILLUSTRATIVE EMBODIMENTS

Magnetic nanoparticles have a wide range of applications both as therapeutic and as diagnostic tools. However, many applications for the particles necessitate the functionalization of the particle surface which can be problematic in the case of pure metals. Likewise, pure metal nanoparticles, while highly effective as hyperthermia inducing agents are prone to oxidation which reduces their specific activity. Likewise, pure metal nanoparticles, absent and effective coating, do not have optimal biocompatibility or circulation kinetics.

The invention addresses current limitations of magnetic nanoparticles and is based in part on use of graphene coated nanoparticles, which exhibit a variety of advantageous properties. First, the graphene surface provides a substrate for functionalization of the nanoparticles. For example, the graphene layer can be functionalized with Poly-γ-glutamic acid (yPGA) and Polyethylene Glycol (PEG) and then conjugated to a therapeutic or targeting moiety (e.g., a nanobody). Likewise, a graphene layer can protect the metal nanoparticle from oxidation. This can be particularly important as pure metal (non-oxidized) nanoparticles can be 6-8 time more effective for hyperthermia therapy. Graphene coating also provides nanoparticles that have excellent biocompatibility, high aqueous solubility and resistance to low and high pH values, all of which are crucial for therapeutic regimes that employ nanoparticles. For example, properly functionalized graphene coated particles (e.g., poly-γ-glutamic acid-methylated polyethylene glycol) can remain in circulation in the serum for more than 18.1 hours. Likewise, the ability of graphene coated particles to disperse in water-based solution and remain intact at low pH combined with the high stability of the targeting moiety (i.e., nanobodies) at low pH allows for the use of the conjugate complex in orally administered formulations, which would not be effective using conventional particles.

As discussed above the nanoparticles of the embodiments are, in certain aspects, conjugated to a targeting moiety, such as an antibody. While it contemplated that a wide range of antibodies may be used as targeting moieties, in preferred aspects the antibody is a single chain antibody or nanobody. In additional to being highly specific to targeting nanoparticles, nanobodies have the advantage of high volume production by, for example, recombinant expression of the nanobody in cells (e.g., utilizing yeast in a bioreactor). Being expressed from a single gene entails maximum reproducibility with minimum encountered mutations. Even more importantly, nanobodies can bind to their targets with a high degree of stability and are resistant to a wide range of pH environments. This allows conjugated nanoparticles to be effectively targeted even after passed through the stomach during oral administration. Likewise, the nanobodies stable interaction with antigen rendering the binding resistant to heating during hyperthermia therapy. For example, whereas a typical antibody-antigen binding interaction will not remain stable above about 45° C., nanobody-antigen binding can remain stable at temperatures of 72° C.

Nanoparticles of the embodiments are ideal for a number for therapeutic applications including as antitumor, anti-bacterial and anti-viral agents. In certain aspects, coated nanoparticles can be used in anti-parasitic therapies. For example, the nanoparticles can comprise a targeting moiety that binds to an antigen (e.g., a protein, lipoprotein or glycoprotein) found on surface membrane or inside (e.g., luminal gut) a parasite. In preferred aspects, the targeted parasite antigen is an antigen expressed in the gut of the parasite. Therapies targeted to parasite gut can for instance, be used to kill the parasite while leaving the exterior of the organism intact. This process thereby avoids the disruption of the exterior of the parasite which could release antigens into that cause adverse reactions in a subject under treatment (e.g., anaphylaxis).

Taken together the advantages offered by the nanoparticles of the embodiments result in more effective therapeutic agents with higher specific activity for per particle. Accordingly effective use of the new particles with require a lower dosage than conventional therapies and should therefore result in fewer and more mild side effects.

### I. Nanoparticles

Nanoparticles according to the embodiments comprise a metal core and a graphene coating encompassing the metallic core. In some embodiments, a nanoparticle core includes at least one metal selected from among scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof. However, in preferred aspects, nanoparticle core comprises a magnetic metal core, even more preferably a substantially non-oxidized metal core.

Metal nanoparticle cores are coated with grapheme using a microwave arc deposition and radio frequency-catalytic chemical vapor deposition (RF-cCVD) (see, Liang et al. 2008 and Biris et al. 2010), which effectively coat particles with aberrant production of carbon nanotubes and fullerenes.

In certain aspects nanoparticles of the embodiments are further coated with molecules for attachment of functional elements (e.g., targeting moieties or therapeutics) or to further improve the biocompatibility of the particles. Examples of such coatings for particles include, but are not limited to, chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin or dextrin or cyclodextrin. In specific aspects, polyglutamic acids (e.g., poly-γ-glutamic acid (γPGA) may used to coat or functionalize a nanoparticle of the embodiments).

Graphene-coated nanoparticles (with or without an additional polymer coating) are conjugated to a targeting moiety as detailed below.

### II. Targeting Moieties

Targeted delivery is achieved by the addition of ligands or other targeting moieties. It is contemplated that this may enable delivery to specific cells, tissues, organs or foreign organisms. The targeting moieties may either be non-covalently or covalently associated with a nanoparticle, and can be conjugated to the nanoparticles by a variety of methods as discussed herein. For example, the nanoparticle may be coupled to a parasite targeting moiety. For example, the target antigen may be a parasite Capthesin B protein, such as Sm31from Schistosoma. Another example antigen for targeting is Capthesin L, such as Capthesin L from Fasciola or Schistosoma species.

In one embodiment, the targeting moiety comprises at least one antibody. The term “antibody” as used herein refers to immunoglobulin molecules and immunologically active portions of immunoglobulin molecules, i.e., molecules that contain an antigen binding site which specifically binds an antigen, such as a polypeptide of the disclosure, e.g., an epitope of a polypeptide of the disclosure. A molecule which specifically binds to a given polypeptide of the disclosure is a molecule which binds the polypeptide, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. The disclosure provides polyclonal and monoclonal antibodies. Synthetic and genetically engineered variants (See U.S. Pat. No. 6,331,415) of any of the foregoing are also contemplated by the present disclosure. Polyclonal and monoclonal antibodies can be produced by a variety of techniques, including conventional murine monoclonal antibody methodology e.g., the standard somatic cell hybridization technique of Kohler and Milstein, Nature 256: 495 (1975) the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. See generally, Harlow, E. and Lane, D. (1988) Antibodies: A Laboratory Manual, Cold Spring Harbor Laboratory Press, Cold Spring Harbor, N.Y.; and Current Protocols in Immunology, Coligan et al. ed., John Wiley & Sons, New York, 1994. Additionally, for use in in vivo applications the antibodies of the present disclosure are preferably human or humanized antibodies. Hybridoma cells producing a monoclonal antibody of the disclosure are detected by screening the hybridoma culture supernatants for antibodies that bind the polypeptide of interest, e.g., using a standard ELISA assay.

Also within the scope of the disclosure, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the disclosure can be selected or (e.g., partially purified) or purified by, e.g., affinity chromatography to obtain substantially purified and purified antibody. By a substantially purified antibody composition is meant, in this context, that the antibody sample contains at most only 30% (by dry weight) of contaminating antibodies directed against epitopes other than those of the desired protein or polypeptide of the disclosure, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the disclosure.

Additionally, recombinant antibodies, such as chimeric and humanized monoclonal antibodies, comprising both human and non-human portions, which can be made using standard recombinant DNA techniques, are within the scope of the disclosure. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671, European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al., (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al., (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al (1986) Nature 321:552-525; Verhoeyan et al. (1988) Science 239:1534; and Beidler et al. (1988) J. Immunol. 141:4053-4060.

Methods for making human antibodies are well known in the art. One method for making human antibodies employs the use of transgenic animals, such as a transgenic mouse. These transgenic animals contain a substantial portion of the human antibody producing genome inserted into their own genome and the animal's own endogenous antibody production is rendered deficient in the production of antibodies.

Antibody fragments may also be derived from any of the antibodies described above. For example, antigen-binding fragments, as well as full-length monomeric, dimeric or trimeric polypeptides derived from the above-described antibodies are themselves useful. Useful antibody homologs of this type include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; (vii) a single domain functional heavy chain antibody, which consists of a VHH domain (known as a nanobody) see e.g., Cortez-Retamozo, et al., Cancer Res. 64. 2853-2857, 2004, Vincke et al., Proc. International Camel Conf. Bikaner, 16-17:71-75, 2007 and De Genst, et al., Dev. And Compar. Immunol., 30:187-198, 2006, each incorporated herein by reference; and (vii) an isolated complementarity determining region (CDR), e.g., one or more isolated CDRs together with sufficient framework to provide an antigen binding fragment. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988); and Huston et al. Proc. Natl. Acad. Sci. USA 85:5879-5883 (1988). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antibody fragments, such as Fv, F(ab′).sub.2 and Fab may be prepared by cleavage of the intact protein, e.g. by protease or chemical cleavage.

Exemplary antibodies (or nanobodies) include those targeting parasite antigens, such as gut antigens of a Fasciolopsis buski, Fasiola hepatica, Opisthorchis sinesis, Paragonimus westermani, Schistosoma species, Taenia species, Diphyllobothrium latum, Echinococcus species, Hymenolepsis species, Enterobius vermicularis, Ascaris lumbricoides, Toxocara species, Trichuris trichiura, Ancylostoma duodenale, Necator americanus, Ancylostoma braziliense, Strongyloides stercoralis, Trichinella spiralis, Wuchereria bancrofti, Brugia malayi, Loa loa, Mansonella species, Onchocerca volvulus, Dirofilaria immitis, Dracunculus medinensis, Plasmodium species, Babesia species, Trypanosoma species, Leishmania species, Toxoplasma species, Sarcocytis species, Acanthamoeba species, Balamuthia species or Naegleria species parasite.

### III. Therapeutic Agents

The nanoparticles of the present invention and formulations thereof may optionally include one or more additional therapeutic agents. For example, the therapeutic agent can be conjugated to the nanoparticle or administered in conjunction with the particles.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristine, vinblastine and methotrexate and pharmaceutically acceptable salts, acids or derivatives of any of the above.

### IV. Administration of Nanoparticle Formulations

The nanoparticles are administered in an amount effective to provide the desired level of biological, physiological, pharmacological and/or therapeutic effect. The nanoparticle may stimulate or inhibit a biological or physiological activity (e.g., of a parasite). The concentration of the nanoparticle should not be so high that the composition has a consistency that inhibits its delivery to the administration site by the desired method. The lower limit of the amount of the nanoparticle may depend on its activity and the period of time desired for treatment.

Where clinical application of the particles of the present invention is undertaken, it will generally be beneficial to prepare the particles as a pharmaceutical composition appropriate for the intended application. This may entail preparing a pharmaceutical composition that is essentially free of pyrogens, as well as any other impurities that could be harmful to humans or animals. One may also employ appropriate buffers to render the complex stable and allow for uptake by target cells.

The phrase “pharmaceutical or pharmacologically acceptable” refers to molecular entities and compositions that do not produce an adverse, allergic or other untoward reaction when administered to an animal, such as a human, as appropriate. For animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound, such as the nanoparticle or the integrated metal radioisotope. In other embodiments, the active compound may comprise between about 1% to about 75% of the weight of the unit, or between about 5% to about 50%, for example, and any range derivable therein. In other non-limiting examples, a dose may also comprise from about <1 microgram/kg/body weight, about 50 microgram/kg/body weight, about 100 microgram/kg/body weight, about 500 microgram/kg/body weight, about 1 milligram/kg/body weight, about 5 milligram/kg/body weight, about 10 milligram/kg/body weight, about 30 milligram/kg/body weight, about 40 milligram/kg/body weight, about 50 milligram/kg/body weight, about 100 milligram/kg/body weight, or more per administration, and any range derivable therein. In non-limiting examples of a derivable range from the numbers listed herein, a range of about 5 microgram/kg/body weight to about 5 milligram/kg/body weight, about 50 microgram/kg/body weight to about 50 milligram/kg/body weight, etc., can be administered.

A nanoparticle may be administered in a dose of 1, 2, 3, 4, 5, 6, 7, 8, 9, 10, 15, 20, 25, 30, 40, 50, 60, 70, 80, 90, 100 or more mg of nanoparticle per dose. Each dose may be in a volume of 1, 10, 50, 100, 200, 500, 1000 or more μl or ml.

Solutions of therapeutic compositions can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The therapeutic compositions of the present invention may include classic pharmaceutical preparations. Administration of therapeutic compositions according to the present invention may be via any common route so long as the target tissue is available via that route. This includes oral, nasal, buccal, rectal, vaginal, topical, or aerosol.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### V. Methods of Using Nanoparticles for Imaging

Nanoparticles may be used in an imaging or detection method for diagnosis or localization of tumor, angiogenic tissues, or bacterial or parasitic infections. Any optical or nuclear imaging method may be contemplated, such as PET, SPECT, CT, MRI or photoacoustic and thermoacoustic tomography. In certain aspects the particles may be conjugated to a radioactive isotope (either for imaging or radiotherapy) or quantum dot fluorescent nanocomposites.

Nanoparticles may be used in PET. Positron emission tomography (PET) is a powerful and widely used diagnostic tool that has the advantages of high sensitivity (down to the picomolar level) and ability to provide quantitative imaging analyses of in vivo abnormalities (Scheinin et al., 1999; Eckelman, 2003; Welch et al., 2009).

Nanoparticles may also be used in SPET. Single photon emission computed tomography (SPECT, or less commonly, SPET) is a nuclear medicine tomographic imaging technique using gamma rays and magnetic resonance imaging. It is very similar to conventional nuclear medicine planar imaging using a gamma camera. However, it is able to provide true 3D information. This information is typically presented as cross-sectional slices through the patient, but can be freely reformatted or manipulated as required.

The SPET basic technique requires injection of a gamma-emitting radioisotope called radionuclide) into the bloodstream of the patient. In certain aspects the radioisotope is integrated into a nanoparticle, which has chemical properties which allow it to be concentrated in ways of medical interest for disease detection. In other aspects, a nanoparticle comprising a marker radioisotope, which is of interest for its radioactive properties, has been attached to a targeting ligand, which is of interest for its chemical binding properties to certain types of tissues. This marriage allows the combination of ligand and radioisotope (the radiopharmaceutical) to be carried and bound to a place of interest in the body, which then (due to the gamma-emission of the isotope) allows the ligand concentration to be seen by a gamma-camera.

Nanoparticles of the embodiments may also be used in conjunction with magnetic resonance imaging (MRI). For example, MRI can be used to visualize targeted nanoparticles to assit in a medical diagnosis or to monitor and nanoparticle-based therapy. Thus, in certain aspects, nanoparticlers of the embodiments additionally comprise an MRI contrast agent. Likewise, MRI may be used to apply nanoparticle based hyperthermia. In this latter aspects a magnetic field is applied having sufficient strength and frequency to facilitate localized heating in tissues comprising the nanoparticles. Methods for using nanopartciles in conjucation with MRI are detailed in Krishman, IEEE Transactions on Magnetics, 46:2523-2558, 2010, incorporated herein by reference.

Nanoparticles may also be used in CT. Computed tomography (CT) is a medical imaging method employing tomography created by computer processing. Digital geometry processing is used to generate a three-dimensional image of the inside of an object from a large series of two-dimensional X-ray images taken around a single axis of rotation. CT is used in medicine as a diagnostic tool and as a guide for interventional procedures. Sometimes contrast materials such as intravenous iodinated contrast are used. This is useful to highlight structures such as blood vessels that otherwise would be difficult to delineate from their surroundings. Using contrast material can also help to obtain functional information about tissues.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Nanoparticle Synthesis

### Iron Nanoparticle Synthesis

Two exemplary protocols for iron nanoparticle synthesis are provided below:

Reaction: 2FeCl₃+6NaBH₄+18H₂O→2Fe(s)+21H₂+6B(OH)₃+6NaCl

1) Create solution A: Dissolve ferric chloride (FeCl₃.6H₂O) in de-ionized water

2) Create solution B: Dissolve sodium borohydride (NaBH₄) in de-ionized water

3) Add solution A to solution B drop wise under vigorous stirring (black particles appear in solution)

4) Remove particles with strong magnet (or magnetic stirrer) or centrifugation

5) Wash particles to remove impurities (de-ionized water or 1:1 ratio of methanol and chloroform or de-ionized water and ethanol)

6) Dry particles (freeze-dry overnight or in vacuum at 60° C. overnight)

Reaction: 4Fe(OH)₃+3N₂H₄ 4 Fe+12H₂O+3N₂

1) Create solution A: Dissolve ferric chloride (FeCl₃.6H₂O) in de-ionized water

2) Add NaOH and N₂H₄.H₂O (80% concentration) to solution A and stir vigorously

4) Remove particles with strong magnet (or magnetic stirrer) or centrifugation

5) Wash particles to remove impurities (de-ionized water or 1:1 ratio of methanol and chloroform or de-ionized water and ethanol)

6) Dry particles (freeze-dry overnight or in vacuum at 60° C. overnight)

In certain aspects reaction may additionally include a noble metal such as Palladium ion to promote particle nucleation. Likewise, reactions may comprise dispersants and surfactants to optimize synthesis.

### Graphene Coating

A focused microwave oven is used to irradiate nanoparticles for graphene coating as described in Liang et al. 2008.

### Targeting Moiety Development

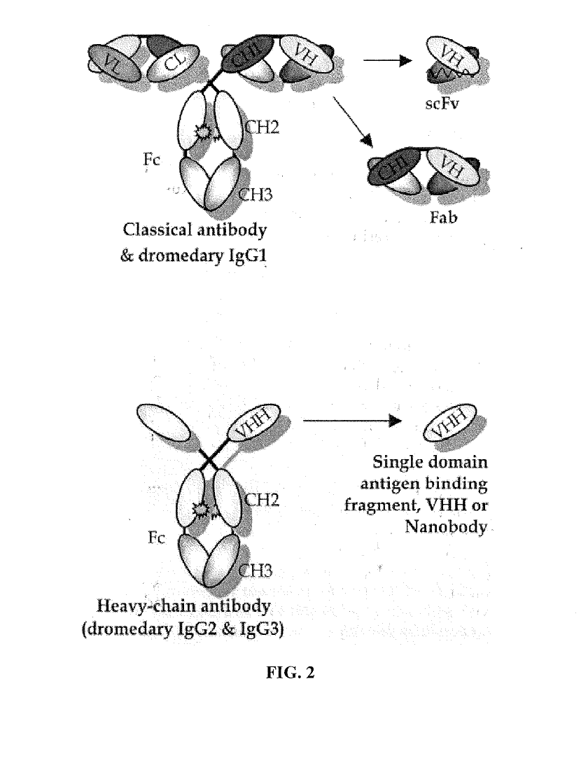

Targeting moieties, such as antibodies, specific for any particular antigen of interest can be produced. For example, nanobodies composed of camelid IgG2 or IgG3 VHH chains can used as targeting moieties (see FIG. 2). These molecules afford high target binding stability and specificity in addition to resistance to low pH and high temperatures.

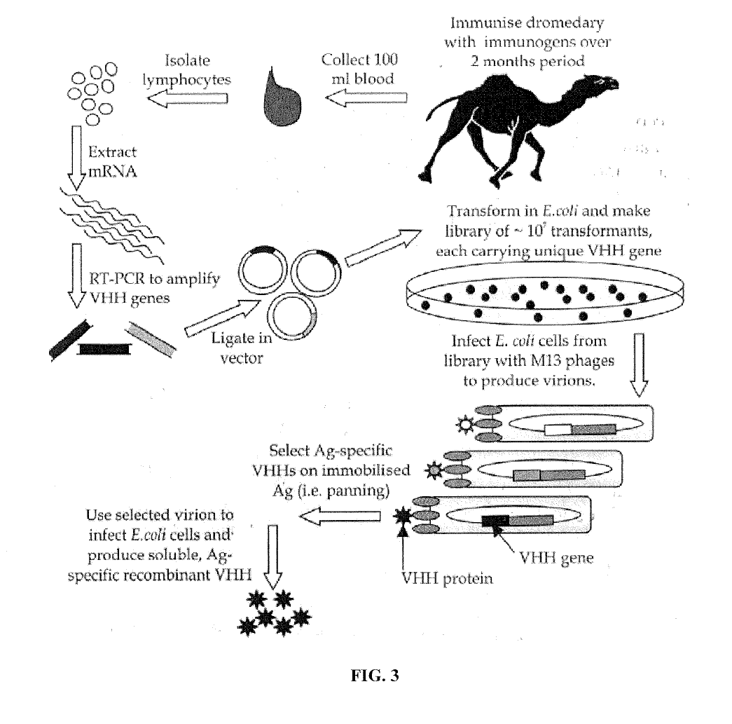

An example protocol for isolating antigen-binding VHH sequences and producing such molecules by recombinant expression is provided in FIG. 3.

### Conjugation to a Targeting Moiety

In case of therapeutics for treating a parasite infection, such as Schistosoma and Fasciola worms infection, nanoparticle can be conjugated to a targeting moiety can binds to a gut antigen in the worm. Targeting moieties that are resistant to heat and acid denaturation (e.g., nanobodies) are preferred such that the targeting moiety can remain intact for both the acidic environment which occurs during oral administration and during heat exposure that occurs during a hyperthermia therapy.

### Example 2

### Radioactive Labelling of Magnetic Beads Conjugated Single Domain Antibodies

1. Introduction

Thanks to their particular properties, single domain antibodies—sdAb have high potential for immuno imaging (1). A technique has recently been developed at the In Vivo Cellular and Molecular Imaging (ICMI) Laboratory of the Nuclear Medicine Department, UZ Brussels, to generate highly specific radiotracers based on sdAb (2). The technique takes advantage of the His-tag that these recombinant molecules contain to form a coordination bond with Tri-carbonyl Technetium [^{99m}Tc(CO)₃(H₂O)₃]⁺. High definition images are obtained with emission tomography (SPECT).

Another interesting technique in imaging is directing magnetised tracers to a specific place of the body, i.e. the organ or sub-organ location of interest, using a focalised magnetic field. In view of a better trespassing of these markers trough physical boarders, we aim to bring this technique to the nano-scale. Therefore, a method to conjugate sdAb to nano scale magnetic beads has been developed at CMIM, VUB. For this purpose, carbon-coated Fe nano magnetic beads of not more than 50 nm and exhibiting functional groups (“Turbobeads”) are conjugated to sdAb. The hereby applied coupling is based on the formation of peptide bonds using water-soluble carbodiimide (CDE) and hydroxysuccinimide (NHS).

This protocol describes the combination of the two techniques: sdAb are conjugated to Turbobeads and labelled with ^{99m}Tc as well.

Tests for the assessment of purity and functionality of the end product are ongoing. This is a living document, initially based on preliminary experiments that provided a first prove of concept in vitro, but adaptable in time as a function of further optimising experiments.

2. Applied Chemistry

2.1 General Principle

The magnetic bead conjugation reaction is based on the formation of peptide bonds by condensation reaction of carboxyl groups situated on the surface of the Turbobeads with the amino groups (Lysine) of the nanobody. On the other hand, the [^{99m}Tc(CO)₃(H₂O)₃]⁺ chelate will be specifically directed to the (His)₆ tag of the nanobody to form a strong coordination bond.

In both reactions, amine groups are involved. Differentiation between Histidine and non-histidine coupling is achieved by conducting two consecutive reactions at different pH. Indeed, the residue of histidine is an imidazole of which the double bound nitrogen atom in the aromatic ring is protonated at pH≦6 (Scheme 1). As a consequence, no nucleophile attack on the carbon of a carboxyl group can take place at pH lower than 6.

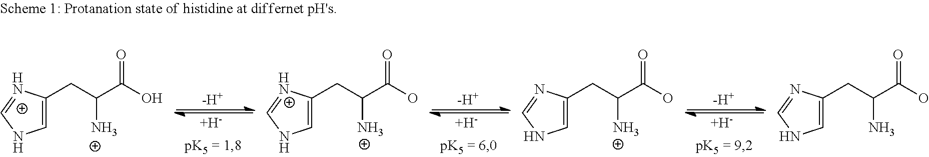

On the contrary, lysine amines are still unprotonated at pH≦6, and peptide bonds between Turbobead bound carboxyl groups and the lysine amino groups of a protein can be formed. The water-soluble carbodiimide (EDC)/N-hydro succinimide (NHS) coupling system has its optimum at pH 5.5.

The hereby presented procedure consists of first performing the condensation reaction at pH 5.5 with the EDC/NHS system, directed to non-histidine (Lys) amines, followed by the His-directed labelling of the nanobody-Turbobead complex with [^{99m}Tc(CO)₃(H₂O)₃]⁺ at pH 7.4 and 50° C. SdAb as well as Turbobeads remain highly stable under in these conditions.

2.2 the Non-his Conjugation of sdAb to Magnetic Beads

This conjugation is performed with a classical condensation reaction using carbodiimide as intermediate, whereby the carboxyl groups of the magnetic beads react with the primary amine groups (terminal or not) of the nanobody to form a peptide bond (scheme 2).

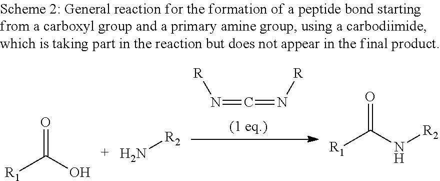

The first intermediate is the unstable o-acyl-isourea, which in the presence of N-hydroxysuccinimide (NHS) is transformed into the corresponding urea and a more stable carboxy-succinimide ester (CSE in scheme 3). The latter reacts spontaneously with primary amines to form the peptide bond.

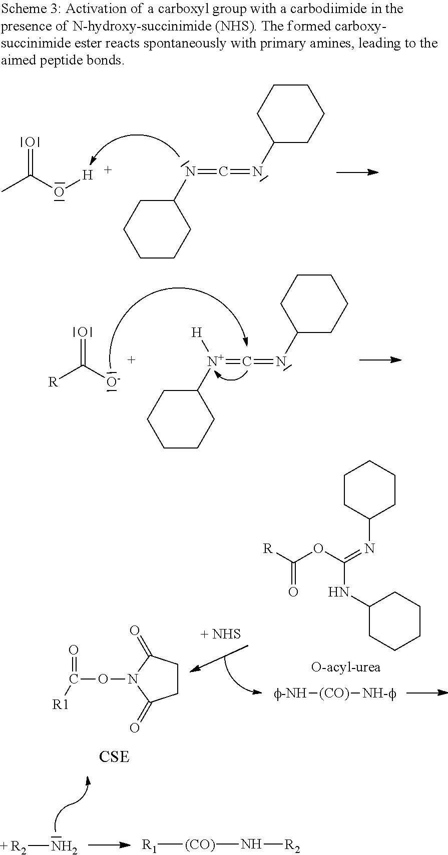

The hereby described procedure activates the carboxyl groups of the magnetic beads with the water soluble 1-Ethyl-3-(3-dimethylaminopropyl) carbodiimide (EDC) and NHS. This system has an optimum at pH 5.5, a condition where histidine does not react.

2.3 the his-Tag Specific Labelling of sdAb with ^{99m}Tc

His-tagged SdAb are particularly suited for ^{99m}Tc-labeling via tricarbonyl-chemistry. Indeed, Histidines have proven to coordinate efficiently to the tricarbonyl core of 99mTc-tricarbonyl (Scheme 4).

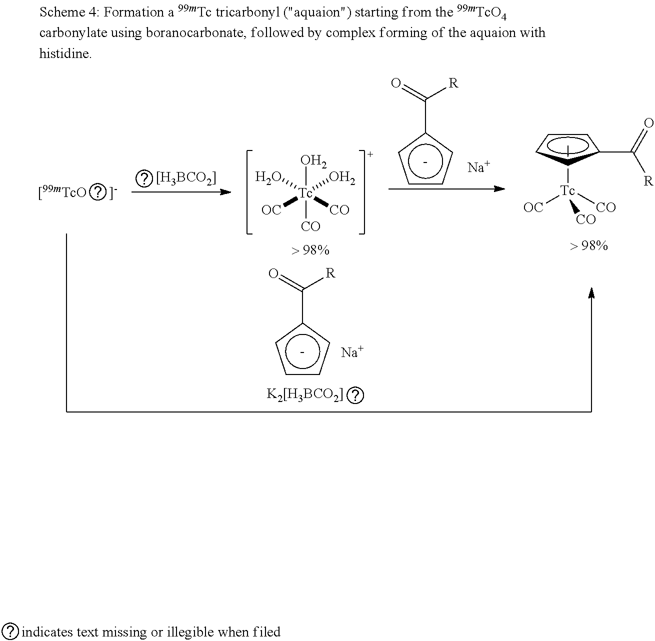

Crystallography studies have also shown that the His-tag on sdAb is located on the opposite side of the paratope(4), hence minimizing the risk of interference with antigen-binding activity. Finally, the high thermal and chemical stabilities have been shown to be essential for efficient complexation of the ^{99m}Tc-tricarbonyl to His-tagged antibody-fragments (5), and these high stability properties are a typical feature of SdAb(6).

The complete labelling procedure has been described by Catarina Xavier et. al (2), and starts with the generation and purity assessment of the ^{99m}Tc-tricarbonyl precursor. This is then followed by a straightforward protocol to complex the precursor with the His-tag of SdAb, and different ways to evaluate the radiochemical purity of the hence obtained Nanobody-derived radiotracer. The Material and methods of this protocol are adapted copies of this procedure.

The [^{99m}Tc(CO)₃(H₂O)₃]⁺ coupling has experimentally been optimised at pH 7.4 and 50° C.

3. Materials and Methods

3.1 General Remark

The protocol was used in studies with turbobeads. One test showed the preservation of functionality of anti-Green Fluorescent Protein (GFP) sdAb after conjugation to turbobeads. Another study produced ^{99m} Tc labelled and turbobeads magnetised sdAb. Migration of the end product towards a magnet was imaged, although functionality was not confirmed.

3.2 Materials

3.2.1. Magnetic Beads Conjugation
Carboxy coated magnetic beads from Turbobeads (Zurich, Switzerland), cat. No 1008, suspended in 5 mL H₂O, 30 mg/mL
MES Buffer: 1.06 g MES (2-(4-Morpholino)ethanesulphonic acid hydrate, e.g. Sigma-Aldrich M 5248) in 90 mL H₂O, adjust to pH 5.5, fill with H₂O to 100 mL
Magnet (alternatively, a pickpen can be used)
EDC solution: Solve 10 mg/mL EDC (Sigma-Aldrich E 7750) in MES buffer
NHS solution: Solve 10 mg/mL NHS (Sigma-Aldrich 56480) in MES buffer (stock in freezer)

3.2.2. His-Tagged sdAb
Highest concentration stock of His-tagged NB in PBS

3.2.3. Preparation of ^{99m} Tc-Tricarbonyl Precursor
Lyophilized kit (IsoLink™, Covidien, St Louis, USA) containing 4.5 mg of sodium boranocarbonate, 2.85 mg of sodium tetraborate.10H₂O, 8.5 mg of sodium tartrate.2H₂O, and 7.15 mg of sodium carbonate, pH 10.5.
Hydrochloric acid (HCl): 1 M solution in water.
⁹⁹Mo/^{99m}Tc generator (Drytec; GE Healthcare).
Well-ventilated hoods and lead shielding.
Water bath or dry heating block.

3.2.4. Assessment of Radiochemical Purity of ^{99m}Tc-Tricarbonyl Precursor
HPLC-system equipped with a radiometric γ-detector.
HPLC column: PLRP-S 300 Å, 5 μm, 250×4.6 mm (Agilent Technologies, Diegem, Belgium).
HPLC solvents: 0.1% trifluoracetic acid (TFA) in H₂O (solvent A) and acetonitrile (solvent B).

3.2.5 Labeling of his-Tagged SdAb with ^{99m}Tc-Tricarbonyl
Nanobody: 1 mg/ml in phosphate buffered saline pH 7.4. (The nanobody solution should be free of imidazole as this substance will interfere with the labeling procedure)
fac-[^{99m}Tc(CO)₃(H₂O)₃]⁺: 0.74-3.7 GBq/ml (from subheading 3.1.)
Eppendorf tubes.
Water bath (50° C.)
Disposable NAP-5 columns (GE Healthcare, Diegem, Belgium), equilibrated with 10 mL-phosphate buffered saline pH 7.4.
0.22 μm membrane filters (4 mm, Millipore, Brussels, Belgium).

3.2.6. HPLC Analysis for Purity Assessment of ^{99m}Tc-Tricarbonyl Nanobody
HPLC-system equipped with a UV and a radiometric γ-detector connected in series.
HPLC column: PLRP-S 300 Å, 5 μm, 250×4.6 mm (Agilent Technologies, Diegem, Belgium).
HPLC solvents: 0.1% trifluoracetic acid (TFA) in H₂O (solvent A) and Acetonitrile (solvent B).

3.2.7 ITLC Analysis for Purity Assessment of ^{99m}Tc-Tricarbonyl Nanobody
Instant Thin Layer Chromatography (ITLC) using silica gel impregnated glass fiber sheets (Pall Corporation, Life Sciences).
ITLC eluent: acetone.
Dose calibrator or gamma counter.

3.3. Methods
3.3.1. Step 1: Conjugation of Magnetic Beads with sdAb

Calculation

Based on the optimisation test, a ratio of approximately 10/1 weight/weight is considered between the magnetic beads and the nanobody. Example: 50 μL magnetic beads suspension corresponds to 1.5 mg beads should be coupled to 150 μg of nanobody.

Sonication

Sonicate magnetic beads in original kit tube during 5 minutes and bring to 25° C.

Washing

Take 50 μL (and another 50 μL if you wish to have a negative control) of magnetic bead suspension and poor into a micro centrifuge tube (2 mL, Sigma-Aldrich, or alternatively polycarbonate) containing 200 μL of MES buffer. Mix gently.

Attract the beads with a magnet to one side of the tube, and eliminate the liquid fraction with a pipette. Add again 200 μL of MES and re-suspend the beads. Repeat 2×, ending with beads suspended in 50 μL MES.

Add EDC: 50 μL EDC solution (1 mL/mL beads) in each tube

Add NHS: 50 μL NHS solution (1 mL/mL beads) in each tube

Incubate for 20 min at room temperature under rotation. N-hydroxysuccinimidez ester activated magnetic beads are formed.

Prepare the nanobody solution In Vivaspin HY 5000 tubes, put 1 mL of MES buffer and, from the highest concentration stock of nanobody, take 150 μg. (for example: if stock=10 mg NB/mL in PBS, take 15 μL) and add to then Vivaspin tube. Concentrate to ±50 and collect this volume.

Isolate the activated beads: Attract the beads with a magnet to one side of the tube, and eliminate the soluble fraction of the reaction mixture.

Add the sdAb: add the nanobody solution to the activated beads and incubate with gentle shaking for 30 minutes.

Collect functionalised beads with the magnet but keep the remaining reaction mixture solution in another tube for testing. Wash the beads with 3λ200 μL PBS/Tween.

3.3.2. Step 2: Labelling with ^{99m}Tc

Preparation of ^{99m}Tc-Tricarbonyl Precursor
Add 1 mL of the ^{99m}TcO₄⁻ solution (⁹⁹Mo/^{99m}Tc generator eluate; 0.74-3.7 GBq) to the IsoLink™ kit.
Incubate the mixture at 100° C. for 20 min.
Cool the reaction mixture in water.
Add HCl 1 M until pH 7.4.

Assessment of Radiochemical Purity of ^{99m}Tc-Tricarbonyl Precursor

For HPLC analysis, inject 2-5 μL of the ^{99m}Tc-Tricarbonyl (3-5 μCi) into the injection loop. Run the following HPLC gradient, at 1 mL/min:
0-5 min: 75% solvent A/25% solvent B
5-7 min: linear gradient of 75% solvent A/25% solvent B to 66% solvent A/34% solvent B
7-10 min: linear gradient of 66% solvent A/34% solvent B to 100% solvent B
10-25 min: 100% solvent B.

The ^{99m}Tc-tricarbonyl precursor shows a retention time of 5-6 min, whereas unreacted ^{99m}TcO₄⁻ shows a retention time of 4 min. Typical purity of [^{99m}Tc(CO)₃(H₂O)₃]⁺ (^{99m}Tc-tricarbonyl) is >95%.

Labeling of His-Tagged SdAb with ^{99m}Tc-Tricarbonyl
Mix 50 μL (50 μg; 1 mg/mL) of purified nanobody with 500 μL of fac-[^{99m}Tc(CO)₃(H₂O)₃]⁺ at pH 7.4.
Incubate at 50° C. for 60-90 min (Temperature of incubation depends on the thermostability of the Nanobody, if possible always determine the melting temperature (Tm) of Nanobody to be labeled).
Separate the labeled nanobody from free ^{99m}Tc-Tricarbonyl and ^{99m}TcO₄⁻ by gel filtration methods such as the NAP-5 column using phosphate buffered saline (If the labeled Nanobody is more lipophilic, there might be some ^{99m}Tc-Tricarbonyl-nanobody activity sticking on the NAP-5 column).
Pass the purified solution through a 0.22 μm membrane filter to eliminate possible aggregates.
Evaluate radiochemical purity by RP-HPLC (see 3.4.1) and/or by ITLC (see 3.4.2). Note that radiochemical purity before gel filtration, as determined by either method, usually ranges from 90 to 98%, and depends on protein concentration. At 0.1 mg/mL final concentration, labelling will be complete after 60 min. After gel filtration and microfiltration, radiochemical purity should be >98% before in vivo assessment.

HPLC Analysis for the Assessment of Radiochemical Purity of ^{99m}Tc-Tricarbonyl Nanobody
Inject 2-5 μL of the ^{99m}Tc-Tricarbonyl Nanobody (3-5 μCi) into the injection loop. Run the following HPLC gradient, at 1 mL/min:
   0-5 min: 75% solvent A/25% solvent B
   5-7 min: linear gradient of 75% solvent A/25% solvent B to 66% solvent A/34% solvent B
   7-10 min: linear gradient of 66% solvent A/34% solvent B to 100% solvent B
      10-25 min: 100% solvent B
The ^{99m}Tc-Tricarbonyl Nanobody shows a retention time of 13 min. The ^{99m}Tc-tricarbonyl precursor shows a retention time of 5-6 min, and ^{99m}TcO₄⁻ has a retention time of 4 min.

ITLC Analysis for Assessment of Radiochemical Purity of ^{99m}Tc-Tricarbonyl Nanobody
Spot 2 μL of ^{99m}Tc-Tricarbonyl Nanobody solution on a 15 mm×200 mm silica gel impregnated glass fiber sheet.
Develop the chromatogram in acetone.
Analyze the distribution of radioactivity by scanning with a γ-radiation TLC scanner or counting the strip cut in 3 parts (application point, middle, solvent front) in a dose calibrator or gamma counter. The ^{99m}Tc-Tricarbonyl precursor and the ^{99m}TcO₄⁻ reveal a Rf (retention factor) of 1 and ^{99m}Tc-Tricarbonyl-nanobody a Rf of 0.

### REFERENCES

The following references, to the extent that they provide exemplary procedural or other details supplementary to those set forth herein, are specifically incorporated herein by reference.
U.S. Patent Publn. No. 20050037421
U.S. Patent Publn. No. 20060211088
U.S. Patent Publn. No. 20100021384
U.S. Patent Publn. No. 20100292564
Cortez-Retamozo, et al., Cancer Res. 64. 2853-2857, 2004.
De Genst, et al., Dev. And Compar. Immunol., 30:187-198, 2006.
Krishman, IEEE Transactions on Magnetics, 46:2523-2558, 2010.
Liang et al., Small, 4:405-409, 2008.
Vincke et al., Proc. International Camel Conf. Bikaner, 16-17:71-75, 2007.
Immuno-imaging using nanobodies. Vaneycken I, D'huyvetter M, Hernot S, De Vos J, Xavier C, Devoogdt N, Caveliers V, Lahoutte T. Curr Opin Biotechnol. 2011 Jul. 2. [Epub ahead of print].
Catarina Xavier1, Nick Devoogdt1, Sophie Hernot1, Ilse Vaneycken1, Matthias D'Huyvetter1, Jens De Vos1,2,3, Sam Massa1,2,3, Tony Lahoutte1,4, Vicky Caveliers1,4 Site-specific labeling of His-tagged nanobodies with 99mTc: a practical guide (submitted paper).
Egli, A., et al., Organometallic 99mTc-aquaion labels peptide to an unprecedented high specific activity. J Nucl Med, 1999. 40(11): p. 1913-7.
Vincke, C., et al., General Strategy to Humanize a Camelid Single-domain Antibody and Identification of a Universal Humanized Nanobody Scaffold. J Biol Chem, 2009. 284(5): p. 3273-84.
Willuda, J., et al., High thermal stability is essential for tumor targeting of antibody fragments: engineering of a humanized anti-epithelial glycoprotein-2 (epithelial cell adhesion molecule) single-chain Fv fragment. Cancer Res, 1999. 59(22): p. 5758-67.
Dumoulin, M., et al., Single-domain antibody fragments with high conformational stability. Protein Sci, 2002. 11(3): p. 500-15.

1. A nanoparticle comprising:
a) a core comprising a magnetic metal;
b) a graphene coating surrounding the core; and
c) a targeting moiety conjugated to the graphene coating.

2. The nanoparticle of claim 1, wherein the magnetic metal is iron, iron-platinum, cobalt, nickel or an oxide of any of the foregoing.

3. (canceled)

4. The nanoparticle of claim 1, wherein the core is greater than 60%, 70%, 80%, 90% or 95% by weight non-oxidized metal.

5. The nanoparticle of claim 1, wherein the core is substantially free of oxidized metal.

6. The nanoparticle of claim 1, wherein the core is greater than 60%, 70%, 80%, 90% or 95% by weight iron.

7. The nanoparticle of claim 1, wherein the core is less than about 20%, 10%, 5%, 3% or 1% by weight iron oxide.

8. The nanoparticle of claim 1, wherein the nanoparticle has an average diameter from about 10 nm to about 500 nm; about 10 nm to about 300 nm; 10 to about 150 nm; about 20 to about 40 nm or about 30 nm.

9. The nanoparticle of claim 1, wherein the graphene coating forms a fullerene structure around the core.

10. The nanoparticle of claim 1, wherein the graphene coating is deposited by microwave arc discharge.

11. The nanoparticle of claim 1, further comprising a therapeutic agent.

12. The nanoparticle of claim 1, wherein the targeting moiety is non-covalently or covalently attached to the nanoparticle.

13. The nanoparticle of claim 1, wherein the targeting moiety is an antibody.

14. The nanoparticle of claim 13, wherein the antibody is an antibody-like molecule, Fc portion, Fab, Fab2, ScFv, a single domain antibody or a nanobody.

15. (canceled)

16. The nanoparticle of claim 1, wherein the targeting moiety binds to a parasite target antigen.

17. The nanoparticle of claim 16, wherein the parasite target antigen is present in the gut of the parasite.

18. The nanoparticle of claim 16, wherein the parasite target gut specific antigen is Capthesin B or Capthesin L.

19. The nanoparticle of claim 16, wherein the parasite is Trematode, Cestode, Nematode or Protozoa parasite.

20. The nanoparticle of claim 19, wherein the parasite is Fasciolopsis buski, Fasiola hepatica, Opisthorchis sinesis, Paragonimus westermani or Schistosoma species.

21. The nanoparticle of claim 1, further comprising a polymer coating.

22. The nanoparticle of claim 21, wherein the polymer is non-covalently or covalently attached to the graphene coating.

23. The nanoparticle of claim 21, wherein the polymer coating is a poly-γ-glutamic acid-methylated polyethylene glycol coating.

24. The nanoparticle of claim 21, wherein a targeting moiety is attached to the polymer coating.

25. A pharmaceutical composition comprising a plurality of nanoparticles according to claim 1 and pharmaceutically acceptable carrier.

26. A method for making a nanoparticle comprising:
a) reducing a metal salt to form a magnetic metal nanoparticle;
b) depositing a graphene coating on the particle by microwave arc discharge; and
c) conjugating the nanoparticle to a targeting moiety.

27. The method of claim 26, wherein the metal salt is an iron salt.

28. The method of claim 26, wherein steps (a) and (b) are performed in concomitantly.

29. The method of claim 26, wherein steps (a) and (b) are performed in the same reaction vessel.

30. The method of claim 26, wherein steps (a) and (b) are performed in the absence of oxygen.

31. The method of claim 26, further comprising coating the nanoparticle with a polymer.

32. The method of claim 26, further comprising attaching a therapeutic to the nanoparticle.

33. The method of claim 26, wherein the targeting moiety is a single domain antibody.

34. A nanoparticle produced by the method of claim 26.

35. A method of treating a subject comprising:
(a) administering nanoparticles comprising a magnetic metal core; a graphene coating and a targeting moiety to a subject; and
(b) applying an alternating current field to the subject, wherein the amount of nanoparticles administered to the subject and the alternating current field applied to the subject are together effective to produce localized hyperthermia in the subject.

36-37. (canceled)

38. The method of claim 35, further defined a methods for treating a bacterial infection, a viral infection, a parasite infection, an autoimmune disease or a cell hyperproliferative disease.

39. (canceled)

40. The method of claim 38, further comprising applying a localized magnetic field to the subject, wherein the field applied to the subject is effective to promote accumulation of nanoparticles in a localized region.

41. A method of treating a subject comprising:
(a) administering nanoparticles comprising a magnetic metal core; a graphene coating and a targeting moiety to a subject;
(b) applying a first magnetic field to the subject, wherein the field applied to the subject is effective to promote accumulation of nanoparticles in a localized region; and
(c) applying an alternating current field to the subject, wherein the amount of nanoparticles administered to the subject and the alternating current field applied to the subject are together effective to produce localized hyperthermia in the subject.

42-44. (canceled)

45. A method for treating a parasitic infection comprising:
(a) administering nanoparticles comprising a magnetic metal core; and a parasite targeting moiety to a subject; and
(b) applying an alternating current field to the subject, wherein the amount of nanoparticles administered to the subject and the alternating current field applied to the subject are together effective to produce hyperthermia at a site of parasite infection in the subject.

46-53. (canceled)
